(19) United States
(12) Patent Application Publication
(10) Pub. No.: US 2025/0090541 A1
(43) Pub. Date: Mar. 20, 2025

(54) Methods for Treating Neurodegenerative Disorders

(71) Applicant: Praetego Inc, Durham, NC (US)

(72) Inventor: Raja Khalifah, Durham, NC (US)

(21) Appl. No.: 18/818,005

(22) Filed: Aug. 28, 2024

Related U.S. Application Data
(63) Continuation of application No. PCT/US2023/066079, filed on Apr. 21, 2023.
(60) Provisional application No. 63/334,149, filed on Apr. 24, 2022.

(51) Int. Cl. A61K 31/55; A61K 31/4439; A61K 31/496; A61K 31/5377; A61P 25/28

(52) U.S. Cl. CPC A61K 31/55; A61K 31/4439; A61K 31/496; A61K 31/5377; A61P 25/28

(57) ABSTRACT

Disclosed are methods for treating or inhibiting development of a disorder selected from the group consisting of a frontotemporal dementia (FTD), Pick's disease, progressive supranuclear palsy, Huntington's disease, Parkinson's disease, corticobasal degeneration, amyotrophic lateral sclerosis (ALS), Lewy body disease, and hippocampal sclerosis (HS), by administering a compound of formula I.

### CROSS REFERENCE

This application is a continuation of PCT/US2023/066079, filed Apr. 21, 2023, which claims the benefit of U.S. Provisional Patent Application Ser. No. 63/334,149, filed Apr. 24, 2022, incorporated by reference herein in its entirety.

### FEDERAL FUNDING STATEMENT

This invention was made with government support from the National Institutes of Health (NIH) under Grant No. R42DK120063 from the National Institute of Diabetes and Digestive and Kidney Diseases (NIDDK), Grant No. 3R42DK120063-02S1 co-funded by the NIDDK with the National Institute on Aging (NIA), and Grant No. R44AG079696 from the NIA. The government has certain rights in the invention.

### SUMMARY

In one aspect, the disclosure provides methods for treating or inhibiting development of a disorder selected from the group consisting of a frontotemporal dementia (FTD), Pick's disease, progressive supranuclear palsy, Huntington's disease, Parkinson's disease, corticobasal degeneration, amyotrophic lateral sclerosis (ALS), Lewy body disease, and hippocampal sclerosis (HS), comprising administering to a subject an amount effective to treat or inhibit development of the disorder of a compound of formula I:

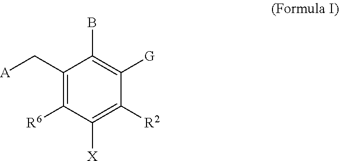

or a pharmaceutically acceptable salt thereof, wherein
X is N, N—O or CR¹;
G is —OH, —SH, —NH₂, or —N(R^{G})₂, wherein R^{G} is hydrogen, (C₁-C₆)alkyl or —C(O)(C₁-C₆)alkyl;
A is

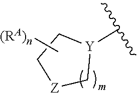

wherein
Y is N;
Z is CH₂, C(H)R^{A}, C(R^{A})₂, O, or NR^{A};
m is 0, 1, 2, or 3;
provided that
   when m is 0, Z is CH₂, C(H)R^{A} or C(R^{A})₂, and
   when m is 2, Z is O or NR^{A};
R^{A} is (C₁-C₆)alkyl, halogen, —OR^{A1}, —N(R^{A1})₂, —SR^{A1}, —S(O)R^{A1}, —S(O)₂R^{A1}, —COOR^{A1}, —CON(R^{A1})₂ or —(C₁-C₆)alkyl-OR^{A1},
   wherein R^{A1} is hydrogen, (C₁-C₆)alkyl or —C(O)(C₁-C₆)alkyl, or two R^{A1} together with N-atom to which they are attached form a morpholinyl; and
n is 0, 1, 2, 3, 4, 5 or 6;
B is of the formula,

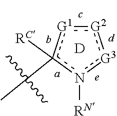

wherein
ring D is (i) monocyclic, and
   (ii) unsaturated or aromatic;
R^{C′} is hydrogen;
G¹ is O, S, N or NR^{N′};
G² and G³ each are independently N, O, CR³, C(R³)₂ or NR^{N′}, wherein each R³ is independently —Z³-M-Z⁴—R^{Z}, wherein M is —C(O)—, —C(S)—, —S(O)—, —S(O)₂—, or absent,
   Z³ and Z⁴ are independently —O—, —S—, —N(R^{N3})— or absent, wherein
      R^{N3} is hydrogen, (C₁-C₆)alkyl, (C₂-C₆)alkenyl, (C₁-C₆)alkynyl, (C₁-C₆)haloalkyl, (C₁-C₆)alkanoyl, (C₃-C₈)cycloalkanoyl, heterocycloyl, aroyl, heteroaroyl, (C₁-C₆)alkoxycarbonyl or aryl(C₁-C₆)alkoxycarbonyl, wherein
      R^{N3} is optionally substituted with one or more groups which are independently halogen, —OH, amino, (C₁—C₆)alkylamino, (C₁-C₆)dialkylamino, —NO₂, —CN, (C₁-C₆)alkyl, aryl, heterocyclyl, heteroaryl, (C₃-C₈)cycloalkyl, (C₁-C₆)haloalkyl, (C₁-C₆)alkoxy, (C₁-C₆)alkanoyl or aroyl;
   R^{Z} is —H, —(C₁-C₆)alkyl, —(C₂-C₆)alkenyl, —(C₁-C₆)alkynyl, or —(C₁-C₆)haloalkyl, —(C₃-C₈)cycloalkyl, —(C₁-C₆)alkylaryl, -heterocycle, -aryl, or -heteroaryl, wherein
      R^{Z} is optionally substituted with at least one R^{Z′}, wherein
      each R^{Z′ is independently -halogen, —OR, —(C}₁-C₆)alkoxy, —C(O)OR, —C(O)R, —C(O)NR₂, —S(O)₂R, —OS(O)₂R, -cyano, -nitro, —(C₁-C₆)alkyl, —(C₁-C₆)haloalkyl, —(C₃-C₈)cycloalkyl, -heterocycloalkyl, or heteroaryl,
   provided when M is —S(O)—, —S(O)₂— or absent, at least one of Z³ and Z⁴ is also absent;
   or two R³ taken together are oxo;
R^{N′} is hydrogen or C₁-C₆)alkyl;
bonds a, b, c, d, and e are independently a single or double bond,
   provided that
   (i) no two consecutive atoms in ring D are both oxygen;
   (ii) no two consecutive bonds are both double bonds;
   (iii) if a or b is a double bond, then R^{C′} is absent; and
   (iv) if a or e is a double bond, then R^{N′} is absent;
   (v) if b or c is a double bond, then G¹ is not O or S;
   (vi) if c or d is a double bond, then G² is not O;
   (vii) if d or e is a double bond, then G³ is not O;
R¹, R², and R⁶ are independently hydrogen, halogen, —NO₂, —CN or R^{C},
   provided that when X═CR¹,
   (i) R², R⁶, and R^{N1} are not phenyl;
   (ii) R^{C} is not aryl, heteroaryl, heterocyclyl or (C₂-C₆)alkenyl
   (iii) and G¹=N together, then G₂ is not O; and
   (iv) two R^{C} together may not form oxo;
and provided that when X═N, and
   (i) G¹ is N, G³ is CR³ and G² is N, and bonds b and d are each a double bond, all simultaneously; or
   (ii) G¹ is N, G³ is C(O), G² is NR^{N′}, and bond b is a double bond, all simultaneously;
either R² or R⁶ is not —NH-aryl or —NH-heteroaryl,
or a pharmaceutically acceptable salt thereof.

In some embodiments, the compound is administered prior to diagnosis of the disorder and the method limits development of the disorder. In other embodiments, the compound is administered subsequent to diagnosis of the disorder and the method treats the disorder. In some embodiments, the method inhibits memory impairment in the subject. In further embodiments, the subject may have mild cognitive impairment, mild dementia, or moderate dementia. In another embodiment, the subject has an amount of total-tau protein and/or phosphorylated tau protein, above control in a cerebrospinal fluid (CSF) sample and/or blood sample; and/or the subject is identified as having pathological intracellular deposits of tau protein by positron emission tomography (PET) imaging. In one embodiment, the subject has a normal level of amyloid β (Aβ) protein in CSF and/or blood samples, and/or the subject is determined to not have pathological Aβ protein deposits by PET imaging.

### DESCRIPTION OF THE FIGURES

FIG. 1. Long-term memory in the Barnes Maze after treatment of htau transgenic mouse with PTG-630 trihydrochloride administered via drinking water at 1 mg/kg/day and 5 mg/kg/day. Unless otherwise specified, the data in these figures refers to male and female mice combined. (A) Mice were tested on the Barnes maze at week 13, then were left 6 weeks without exposure to the maze before being tested again after 19 weeks of treatment with PTG-630. (B) Mice were tested on the Barnes maze at week 19, then were left 3 weeks without exposure to the maze before being tested again after 23 weeks of treatment with PTG-630. *p<0.05, by 1-way ANOVA followed by Holm-Sidak's post hoc test.

FIG. 2. Recognition index after treatment with PTG-630 trihydrochloride. Human tau mice develop significant short-term episodic memory that was partially prevented by treatment with PTG-630, as represented by the recognition index, in htau mice exposed to a new object in the Object Recognition Test. *p<0.05, **p<0.01 by repeated measures ANOVA followed by Tukey's post hoc test.

FIG. 3. Protein levels in hippocampus homogenate from female htau mice for the CNS biomarkers measured by Western blotting: Drebrin (A), PSD95 (B) and NeuN (C). N=6-7, Mean±sem. *p<0.05 one-way ANOVA followed by Dunnet's post-hoc test.

FIG. 4. Tactile allodynia response, a measure of peripheral nervous system (PNS) damage, observed over the 24 weeks of treatment of htau mice with PTG-630 trihydrochloride. *p<0.0001 by repeated measures ANOVA followed by Holm-Sidak's post hoc test.

FIG. 5. Thermal responses to heat after 24 weeks of treatment with PTG-630 trihydrochloride showing amelioration of hypoalgesia damage to peripheral nervous system (PNS). *p<0.05, ***p<0.001, by repeated measures ANOVA followed by Holm-Sidak's's post hoc test.

FIG. 6. Intraepidermal nerve fiber (IENF) changes in male (A) and female (B) mice footskin after 24 weeks of treatment with PTG630 trihydrochloride. *p<0.05, by repeated measures ANOVA followed by Holm-Sidak's's post hoc test.

FIG. 7. 4-Hydroxynonenal levels, a measure of oxidative stress and lipid peroxidation, in hippocampus homogenates from htau mice treated for 25 weeks with PTG-630 trihydrochloride. **p<0.001 by 1-way ANOVA followed by Holm-Sidak post hoc test.

FIG. 8. Area under the curve (AUC) of the time to find the escape box for 5 consecutive days for female 3×Tg mice after 18 weeks of treatment with PTG-670 trihydrochloride administered via drinking water at doses of 1 mg/kg/day and 5 mg/kg/day. *p<0.05 by one way ANOVA followed by Holm-Sidak's post hoc test.

FIG. 9. Long-term memory in the Barnes Maze after treatment with PTG-670 trihydrochloride for female 3×Tg transgenic mouse. Mice were tested on the Barnes maze at week 12, then were left 6 weeks without exposure to the maze before being tested again after 18 weeks of treatment with PTG-670. **p<0.01, by 1-way ANOVA followed by Holm-Sidak's post hoc test.

FIG. 10. Recognition index after 6 to 18 weeks of treatment with PTG-670 trihydrochloride. 3×Tg mice develop significant short-term episodic memory that was alleviated by treatment with PTG-670, *p<0.05 by 2 way ANOVA followed by Holm-Sidak's post hoc test against control and #p<0.05 against 3×Tg+5 mg/kg PTG-670.

FIG. 11. 4-Hydroxynonenal levels, a measure of oxidative stress and lipid peroxidation, in hippocampus homogenates from 3×Tg mice after 20 weeks of treatment with PTG-670 trihydrochloride.

FIG. 12. Protein levels in hippocampus homogenates for the CNS biomarkers Drebrin (A), PSD95 (B) and NeuN (C), measured by Western blotting, in female 3×Tg mice treated for 25 weeks with PTG-670 trihydrochloride. N=6-7, Mean±sem. *p<0.05 one-way ANOVA followed by Holm-Sidak's post-hoc test.

### DETAILED DESCRIPTION OF THE DISCLOSURE

All references cited are herein incorporated by reference in their entirety. As used herein, the singular forms “a”, “an” and “the” include plural referents unless the context clearly dictates otherwise. All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides methods for treating or inhibiting development of a disorder selected from the group consisting of a frontotemporal dementia (FTD; including but not limited to behavioral-variant FTD, primary progressive aphasia, Pick's disease, corticobasal degeneration and progressive supranuclear palsy), Huntington's disease, Parkinson's disease (PD), amyotrophic lateral sclerosis (ALS), Lewy body disease, and hippocampal sclerosis (HS), comprising administering to a subject an amount effective to treat or inhibit development of the disorder of a compound of the formula (I),

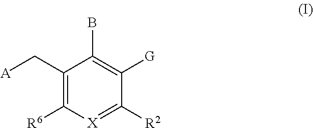

or a pharmaceutically acceptable salt thereof, wherein
X is N, N—O or CR¹;
G is —OH, —SH, —NH₂, or —N(R^{G})₂, wherein R^{G} is hydrogen, (C₁-C₆)alkyl or —C(O)(C₁-C₆)alkyl;
A is

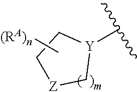

wherein
Y is N;
Z is CH₂, C(H)R^{A}, C(R^{A})₂, O, or NR^{A};
m is 0, 1, 2, or 3;
provided that
   when m is 0, Z is CH₂, C(H)R^{A} or C(R^{A})₂, and
   when m is 2, Z is O;
R^{A} is (C₁-C₆)alkyl, halogen, —OR^{A1}, —N(R^{A1})₂, —SR^{A1}, —S(O)R^{A1}, —S(O)₂R^{A1}, —COOR^{A1}, —CON(R^{A1})₂ or —(C₁-C₆)alkyl-OR^{A1},
   wherein R^{A1} is hydrogen, (C₁-C₆)alkyl or —C(O)(C₁-C₆)alkyl, or two R^{A1} together with N-atom to which they are attached form a morpholinyl; and
n is 0, 1, 2, 3, 4, 5 or 6;
B is of the formula,

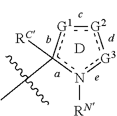

wherein
ring D is (i) monocyclic, and
   (ii) unsaturated or aromatic;
R^{C′} is hydrogen;
G¹ is O, S, N or NR^{N′};
G² and G³ each are independently N, O, CR³, C(R³)₂ or NR^{N′}, wherein each R³ is independently —Z³-M-Z⁴—R^{Z}, wherein M is —C(O)—, —C(S)—, —S(O)—, —S(O)₂—, or absent,
   Z³ and Z⁴ are independently —O—, —S—, —N(R^{N3})— or absent, wherein
      R^{N3} is hydrogen, (C₁-C₆)alkyl, (C₂-C₆)alkenyl, (C₁-C₆)alkynyl, (C₁-C₆)haloalkyl, (C₁-C₆)alkanoyl, (C₃-C₈)cycloalkanoyl, heterocycloyl, aroyl, heteroaroyl, (C₁-C₆)alkoxycarbonyl or aryl(C₁-C₆)alkoxycarbonyl, wherein
      R^{N3} is optionally substituted with one or more groups which are independently halogen, —OH, amino, (C₁-C₆)alkylamino, (C₁-C₆)dialkylamino, —NO₂, —CN, (C₁-C₆)alkyl, aryl, heterocyclyl, heteroaryl, (C₃-C₈)cycloalkyl, (C₁-C₆)haloalkyl, (C₁-C₆)alkoxy, (C₁-C₆)alkanoyl or aroyl;
   R^{Z} is —H, —(C₁-C₆)alkyl, —(C₂-C₆)alkenyl, —(C₁-C₆)alkynyl, or —(C₁-C₆)haloalkyl, —(C₃-C₈)cycloalkyl, —(C₁-C₆)alkylaryl, -heterocycle, -aryl, or -heteroaryl, wherein
      R^{Z} is optionally substituted with at least one R^{Z′}, wherein
      each R^{Z′ is independently -halogen, —OR, —(C}₁-C₆)alkoxy, —C(O)OR, —C(O)R, —C(O)NR₂, —S(O)₂R, —OS(O)₂R, -cyano, -nitro, —(C₁-C₆)alkyl, —(C₁-C₆)haloalkyl, —(C₃-C₈)cycloalkyl, -heterocycloalkyl, or heteroaryl,
   provided when M is —S(O)—, —S(O)₂— or absent, at least one of Z³ and Z⁴ is also absent;
   or two R³ taken together are oxo;
R^{N′} is hydrogen or C₁-C₆)alkyl;
bonds a, b, c, d, and e are independently a single or double bond,
   provided that
   (i) no two consecutive atoms in ring D are both oxygen;
   (ii) no two consecutive bonds are both double bonds;
   (iii) if a or b is a double bond, then R^{C′} is absent; and
   (iv) if a or e is a double bond, then R^{N′} is absent;
   (v) if b or c is a double bond, then G¹ is not O or S;
   (vi) if c or d is a double bond, then G² is not O;
   (vii) if d or e is a double bond, then G³ is not O;
R¹, R², and R⁶ are independently hydrogen, halogen, —NO₂, —CN or R^{C},
   provided that when X═CR¹,
   (v) R², R⁶, and R^{N}i are not phenyl;
   (vi) R^{C} is not aryl, heteroaryl, heterocyclyl or (C₂-C₆)alkenyl
   (vii) and G¹=N together, then G₂ is not O; and
   (viii) two R^{C} together may not form oxo;
and provided that when X═N, and
   (i) G¹ is N, G³ is CR³ and G² is N, and bonds b and d are each a double bond, all simultaneously; or
   (ii) G¹ is N, G³ is C(O), G² is NR^{N′}, and bond b is a double bond, all simultaneously;
either R² or R⁶ is not —NH-aryl or —NH-heteroaryl,
or a pharmaceutically acceptable salt thereof.

While not being limited by any specific mechanism of action, in the examples that follow, the inventor has shown that the methods disclosed herein inhibit memory impairment in an animal model of a tau-protein based neurological disorder, demonstrating the utility of the methods disclosed herein.

Primary tauopathies are a group of neurodegenerative diseases characterized by pathological intracellular deposits of the protein tau. Isoform composition, morphology and anatomical distribution of cellular tau-immunoreactivities are defining distinct primary tauopathies as molecular pathological disease entities. The clinical spectrum of primary tauopathies includes frontotemporal dementia (FTD; including but not limited to behavioral-variant FTD, primary progressive aphasia, Pick's disease, corticobasal degeneration and progressive supranuclear palsy), Huntington's disease, Parkinson's disease (PD), amyotrophic lateral sclerosis (ALS), Lewy body disease, and hippocampal sclerosis (HS).

In one embodiment, the disorder comprises FTD, which includes dementias such as behavioral-variant FTD, primary progressive aphasia, Pick's disease, corticobasal degeneration and progressive supranuclear palsy. In FTD, nerve cells in the front (frontal lobe) and side regions (temporal lobes) of the brain are especially affected and become markedly atrophied. In addition, upper layers of the cortex typically become soft and spongy and abnormal tau protein and/or transactive response DNA-binding protein (TDP-43) is present. The symptoms of FTD may occur in those age 65 years and older, but most people with FTD develop symptoms at a younger age. Typical early symptoms include marked changes in personality and behavior and/or difficulty with producing or comprehending language.

Behavioral-variant FTD is the most common FTD. This variant is characterized by progressive atrophy in frontal and anterior temporal regions of the brain leading to alterations in complex thinking, personality and behavior. Symptoms include behavioral disinhibition (i.e.: socially inappropriate behavior (e.g., inappropriately approaching or touching strangers), loss of manners or decorum (e.g., violation of personal space, rude or offensive remarks), or impulsive, rash or careless actions (e.g., reckless buying or selling)); apathy/inertia, loss of empathy, perseverative/compulsive behaviors (for example, simple repetitive behaviors such as tapping, scratching or picking, to complex compulsive behaviors such ordering, cleaning or collecting); changes in eating habits; and executive dysfunction (i.e.: dysfunction in “executive functions” such as planning, organizing, mental flexibility and generation of ideas).

Primary progressive aphasia affects the ability to communicate and can cause trouble expressing thoughts and understanding or finding words. Symptoms begin gradually, often before age 65, and worsen over time. People with primary progressive aphasia can lose the ability to speak and write and, eventually, to understand written or spoken language.

Pick's disease affects parts of the brain that control emotions, behavior, personality, and language, typically caused by tau protein clumping. Symptoms of Pick's disease include, but are not limited to abrupt mood changes, compulsive or inappropriate behavior, depression-like symptoms, such as disinterest in daily activities; withdrawal from social interaction, difficulty keeping a job, poor social skills, poor personal hygiene, and/or repetitive behavior.

Corticobasal degeneration (CBD) is a rare progressive neurological disorder characterized by cell loss and deterioration of specific areas of the brain. Affected individuals often initially experience motor abnormalities in one limb that eventually spreads to affect all the arms and legs. Such motor abnormalities include muscle rigidity and the inability to perform purposeful or voluntary movements (apraxia). Other symptoms may include, but are not limited to alien limb syndrome (affected individuals may be unaware of the movement of a limb or unable to control the movement of a limb), tremors, exaggerated slowness of movements (bradykinesia) or lack of movement (akinesia), involuntary muscle spasms that cause jerky movements (myoclonus), limb dystonia (involuntary muscle contractions that force a certain part(s) of the body into abnormal, sometimes painful, movements and positions), contractures (a joint becomes permanently fixed in a bent (flexed) or straightened (extended) position), difficulty understanding or expressing language (aphasia), difficulty saying what they want to say despite knowing the right words (apraxia of speech), speech difficulties due to problems with the muscles that enable speech (dysarthria), difficulty swallowing (dysphagia), an inability to control eyelid blinking, and/or an uncoordinated walk (ataxic gait).

Progressive supranuclear palsy (PSP) is a late-onset degenerative disease involving the gradual deterioration and death of specific volumes of the brain, leading to symptoms including loss of balance, slowing of movement, difficulty moving the eyes, and cognitive impairment. PSP typically involves accumulation of tau protein within the brain.

In another embodiment, the disorder comprises Huntington's disease (HD), caused by a mutation in the huntingtin gene that causes neurons to die in various areas of the brain, including those that help to control voluntary (intentional) movement. Symptoms of HD include but are not limited to uncontrolled movements (chorea), abnormal body postures, and changes in behavior, emotion, judgment, and cognition. People with HD also develop impaired coordination, slurred speech, and difficulty feeding and swallowing. HD typically begins between ages 30 and 50. An earlier onset form called juvenile HD occurs under age 20.

In a further embodiment, the disorder comprises Parkinson's disease (PD). In PD, clumps of the protein alpha-synuclein appear in an area deep in the brain called the substantia nigra. These clumps are thought to cause degeneration of the nerve cells that produce dopamine. As PD progresses, alpha-synuclein can also accumulate in the cortex of the brain.

Dementia may result. Symptoms include, but are not limited to, problems with movement (slowness, rigidity, tremor and changes in gait), with cognitive symptoms developing later in the disease, years after movement symptoms.

In one embodiment, the disorder comprises ALS, also known as Lou Gehrig's disease.

In ALS, motor neurons degenerate and die, resulting in the inability to control muscle movement. Patients with ALS may lose their ability to move, speak, eat, or breathe.

Symptoms of ALS include but are not limited to difficulty walking or doing normal daily activities, tripping and falling, weakness in legs, feet or ankles; hand weakness or clumsiness, slurred speech or trouble swallowing, muscle cramps and twitching in arms, shoulders and/or tongue; inappropriate crying, laughing or yawning; cognitive changes, and behavioral changes.

In one embodiment, the disorder comprises Lewy body Disease. Lewy bodies are abnormal aggregations of the protein alpha-synuclein in neurons. When they develop in the cortex, dementia can result (i.e. dementia with Lewy bodies (DLB)). Symptoms of DLB include, but are not limited to sleep disturbances, well-formed visual hallucinations and visuospatial impairment, problems with motor function, and memory loss.

In one embodiment, the disorder comprises hippocampal sclerosis (HS), the shrinkage and hardening of tissue in the hippocampus of the brain. Symptoms of HS include, but are not limited to, seizures and cognitive deficits that relate to the hippocampus affected (verbal memory impairment in dominant hippocampal sclerosis, and visual memory impairment in non-dominant hippocampal sclerosis.

In one embodiment, the compound is administered prior to diagnosis of the disorder and the method limits development of the disorder. Limiting development of the disorder means to limit development (compared to no treatment) of one or more symptoms of the disorder noted herein. Any suitable limitation of development of the symptoms provides a benefit and is thus contemplated herein (i.e.: 5%, 10%, 15%, 20%, 25%, 50%, 75%, or greater reduction in symptoms compared to no treatment). In this embodiment, the subject has not yet suffered the disorder, but is any subject at risk of the disorder, based on age, family history, or any other factors. In these subjects, the compound may be administered for any suitable time period prior to a subject at risk of the disorder.

In another embodiment, the compound is administered subsequent to diagnosis of the disorder and the method treats the disorder. As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

In one specific embodiment, the methods inhibit memory impairment in the subject.

In one embodiment, the subject has a two or more of the disorders. As will be understood by those of skill, subjects will often present with two or more of the recited disorders (i.e.: mixed disorders).

The subject may be any subject susceptible or having one of the disorders, including but not limited to a mammalian subject. In one embodiment, the subject is a human subject.

All of the disorders may present with cognitive impairment or dementia. In one embodiment, the subject has mild cognitive impairment. In another embodiment, the subject has mild dementia. In a further embodiment, the subject has moderate dementia.

As used herein, “mild cognitive impairment” is the stage between the expected cognitive decline of normal aging and the more serious decline of dementia, characterized by problems with memory, language, thinking or judgment.

As used herein, mild dementia is present when the subject is able to function independently in many areas but requires assistance with some activities to maximize independence and remain safe, including but not limited to handling money and paying bills, completing common daily tasks, drive, etc.

As used herein, moderate dementia is present when the subject experiences more problems with memory and language, is more likely to become confused, and finds it harder to complete multistep tasks such as bathing and dressing. They may become incontinent at times, and they may start having personality and behavioral changes.

In another embodiment, the subject has an amount of total-tau protein and/or phosphorylated tau protein (Ttau and P-tau, respectively), above control levels (such as from a normal individual without a tau-based neurological disorder, or standard developed from such a control population) in a cerebrospinal fluid (CSF) sample and/or blood sample; and/or the subject is identified as having pathological intracellular deposits of tau protein by positron emission tomography (PET) imaging.

In another embodiment, the subject has a normal level (relative to a control from subjects not having a neurological disorder) of amyloid β (Aβ) protein in CSF and/or blood samples, and/or the subject is determined to not have pathological Aβ protein deposits by PET imaging.

In another embodiment, the methods comprise administering an effective amount of a compound of formula I, or a pharmaceutically acceptable salt thereof, to treat Alzheimer's disease characterized by pathological intracellular deposits of the protein tau.

In certain embodiments, the compounds of formula (I) are wherein
Z is CH₂, C(H)R^{A}, C(R^{A})₂, or O;
m is 0 or 2;
provided that
   when m is 0, Z is CH₂, C(H)R^{A} or C(R^{A})₂, and
   when m is 2, Z is O.

In another embodiment, the disclosure provides compounds of formula (I) wherein X is N and G is hydrogen.

In another embodiment, the disclosure provides compounds of formula (I) wherein
B is aromatic;
G¹ is O, S, N or NR^{N′}; and
G² and G³ are each independently O, N or CR³.

In another embodiment, the disclosure provides compounds of formula (I) wherein B is imidazolyl, oxazoyl, pyrazoyl, pyrroyl or isoxazoyl wherein each carbon atom is substituted by R³.

In another embodiment, the disclosure provides compounds of formula (I) wherein B is imidazolyl wherein each carbon atom is substituted by R³.

In another embodiment, the disclosure provides compounds of formula (I) wherein
each R³ is independently R^{Z3}, wherein
   R^{Z3} is hydrogen, (C₁-C₆)alkyl, (C₁-C₆)haloalkyl, (C₃-C₈)cycloalkyl, (C₁-C₆)alkylaryl, heterocyclyl, aryl or heteroaryl, wherein RZ3 is optionally substituted with at least one R^{Z3}′, wherein
      each R^{Z3′} is independently halogen, cyano, —OR, —C(O)OR, —C(O)R, —C(O)NR₂, (C₁-C₆)alkyl, (C₁-C₆)haloalkyl, (C₃-C₈)cycloalkyl or heterocycloalkyl, wherein each R is independently hydrogen, (C₁-C₆)alkyl or (C₁-C₆)haloalkyl.

In another embodiment, the disclosure provides compounds of formula (I) wherein
R² and R⁶ are each hydrogen, halogen, —NO₂, —CN or R^{Z6} wherein
   R^{Z6} is (C₁-C₆)alkyl, (C₁-C₆)haloalkyl, (C₃-C₈)cycloalkyl, (C₁-C₆)alkylaryl, heterocyclyl, aryl or heteroaryl, wherein R^{Z6} is optionally substituted with at least one RZ^{6′}
      wherein each R^{Z6′} is independently halogen, —OR, —C(O)OR, —C(O)R, (C₁-C₆)alkyl or (C₁-C₆)haloalkyl, wherein each R is independently hydrogen, (C₁-C₆)alkyl or (C₁-C₆)haloalkyl.

In another embodiment, the disclosure provides compounds of formula (I) wherein
R^{N′} is hydrogen, (C₁-C₆)alkyl, (C₁-C₆)haloalkyl, (C₁-C₆)alkanoyl, (C₃-C₈)cycloalkyl, aryl, heteroaryl, (C₃-C₈)cycloalkanoyl, heterocycloyl, aroyl, heteroaroyl, (C₁-C₆)alkoxycarbonyl or aryl(C₁-C₆)alkoxycarbonyl, wherein
   R^{N′} is optionally substituted with one or more groups which are independently halogen, —OR^{N″}, —NR^{N″}₂, —NO₂, —CN, (C₁-C₆)alkyl, aryl, heterocyclyl, heteroaryl, (C₃-C₈)cycloalkyl or (C₁-C₆)haloalkyl,
      wherein each R^{N″} is independently hydrogen, (C₁-C₆)alkyl, (C₁-C₆)haloalkyl, (C₁-C₆)alkoxy, (C₃-C₈)cycloalkyl, heterocycloalkyl, aryl or heteroaryl.

In another embodiment, the disclosure provides compounds of formula (I) wherein A is

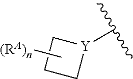

wherein n is 0, 1, 2 or 3.

In another embodiment, the disclosure provides compounds of formula (I) wherein A is

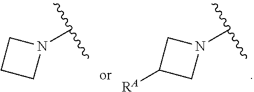

In another embodiment, the disclosure provides compounds of formula (I) wherein A is

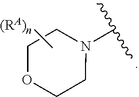

In another embodiment, the disclosure provides compounds of formula (I) wherein R^{A} is (C₁-C₆)alkyl, halogen or —(C₁-C₆)alkyl-OR^{A1}, wherein R^{A1} is hydrogen or (C₁-C₆)alkyl.

In another embodiment, the disclosure provides compounds of formula (I) wherein R^{A} is (C₁-C₆)alkyl, halogen, (C₁-C₆)alkyl-OR^{A1}, or —COOR^{A1}, wherein R^{A1} is hydrogen or (C₁-C₆)alkyl, or two R^{A1} together with N-atom to which they are attached form a morpholinyl.

In another embodiment, the disclosure provides compounds of formula (I) wherein B is not aromatic and G¹, G², and G³ are each independently O, N, CR³, C(R³)₂ or N(R^{N′}).

In another embodiment, the disclosure provides compounds of formula (I) wherein
B is pyrrolidinyl, pyrazolidinyl, imidazolidinyl, isoxazolidinyl, oxazolidinyl, thiazolidinyl or tetrazolidinyl, wherein each carbon is substituted by two R³ and each nitrogen is substituted by R^{N′}.

In another embodiment, the disclosure provides compounds of formula (I) wherein
each R³ is independently R^{Z3}, wherein
   R^{Z3} is hydrogen, (C₁-C₆)alkyl, (C₁-C₆)haloalkyl, (C₃-C₈)cycloalkyl, (C₁-C₆)alkylaryl, heterocyclyl, aryl or heteroaryl, wherein R^{Z3} is optionally substituted with at least one R^{Z3′}, wherein
      each R^{Z3′} is independently -halogen, -cyano, —OR, —C(O)OR, —C(O)R, —C(O)NR₂, (C₁-C₆)alkyl, (C₁-C₆)haloalkyl, (C₃-C₈)cycloalkyl or heterocycloalkyl.

In another embodiment, the disclosure provides compounds of formula (I) wherein
R² and R⁶ are each hydrogen, halogen, —NO₂, —CN or —R^{Z6} wherein
   R^{Z6} is (C₁-C₆)alkyl, (C₁-C₆)haloalkyl, (C₃-C₈)cycloalkyl, (C₁-C₆)alkylaryl, heterocyclyl, aryl or heteroaryl, wherein R^{Z6} is optionally substituted with at least one R^{Z6′},
      wherein each R^{Z6′} is independently halogen, —OR, —C(O)OR, —C(O)R, (C₁-C₆)alkyl or (C₁-C₆)haloalkyl,
         wherein R^{Z6′} is optionally substituted with one or more R′.

In another embodiment, the disclosure provides compounds of formula (I) wherein
each R^{N′} is hydrogen, (C₁-C₆)alkyl, (C₁-C₆)haloalkyl, (C₁-C₆)alkanoyl, (C₃-C₈)cycloalkyl, aryl, heteroaryl, (C₃-C₈)cycloalkanoyl, heterocycloyl, aroyl, heteroaroyl, (C₁-C₆)alkoxycarbonyl or aryl(C₁-C₆)alkoxycarbonyl, wherein
   R^{N′} is optionally substituted with one or more groups which are independently halogen, —OR^{N″}, —NR^{N″}₂, —NO₂, —CN, (C₁-C₆)alkyl, aryl, heterocyclyl, heteroaryl, (C₃-C₈)cycloalkyl or —(C₁-C₆)haloalkyl,
      wherein each R^{N″} is independently hydrogen, (C₁-C₆)alkyl, (C₁-C₆)haloalkyl, (C₁-C₆)alkoxy, (C₃-C₈)cycloalkyl, heterocycloalkyl, aryl or heteroaryl, wherein the alkyl and alkoxy are optionally substituted with one or more R′.

In another embodiment, the disclosure provides compounds of formula (I) wherein X is CR¹ and G is hydrogen.

In another embodiment, the disclosure provides compounds of formula (I) wherein
R¹ is —CN, —NO₂, halogen, —C(O)OR⁴, —C(O)R⁴, —C(O)N(R⁴)₂, —S(O)R⁴, —S(O)₂R⁴ or —S(O)₂N(R⁴)₂, wherein
   each R⁴ is independently hydrogen, (C₁-C₆)alkyl, (C₂-C₆)alkenyl, (C₃-C₈)cycloalkyl, (C₁-C₆)alkylaryl, heterocyclyl, aryl or heteroaryl, wherein
      R⁴ is optionally substituted with at least one group, each of which are independently halogen, —OH, (C₁-C₆)alkoxy, —C(O)R⁴¹, —S(O)₂R⁴¹, —OS(O)₂R⁴¹, —CN, —NO₂, (C₁-C₆)alkyl or (C₁-C₆)haloalkyl,
         wherein R⁴¹ is hydrogen, (C₁-C₆)alkyl, (C₁-C₆)haloalkyl, (C₁-C₆)alkoxy, (C₃-C₈)cycloalkyl, heterocycloalkyl, aryl or heteroaryl.

In another embodiment, the disclosure provides compounds of formula (I) wherein
B is aromatic; and
G¹ is O, S, N or NR^{N′}; and
G² and G³ are each independently O, N or CR³.

In another embodiment, the disclosure provides compounds of formula (I) wherein B is imidazolyl, oxazoyl, pyrazoyl, pyrroyl or isoxazoyl wherein each carbon atom is substituted by R³.

In another embodiment, the disclosure provides compounds of formula (I) wherein B is imidazolyl wherein each carbon atom is substituted by R³.

In another embodiment, the disclosure provides compounds of formula (I) wherein
each R³ is independently R^{Z3}, wherein
   R^{Z3} is hydrogen, (C₁-C₆)alkyl, (C₁-C₆)haloalkyl, (C₃-C₈)cycloalkyl, (C₁-C₆)alkylaryl, heterocyclyl, aryl or heteroaryl, wherein R^{Z3} is optionally substituted with at least one R^{Z3′}, wherein
      each R^{Z3′} is independently halogen, —CN, —OR, —C(O)OR, —C(O)R, —C(O)NR₂, (C₁-C₆)alkyl, (C₁-C₆)haloalkyl, (C₃-C₈)cycloalkyl or heterocycloalkyl.

In another embodiment, the disclosure provides compounds of formula (I) wherein
R² and R⁶ are each hydrogen, halogen, —NO₂, —CN or —R^{Z6} wherein
   R^{Z6} is (C₁-C₆)alkyl, (C₁-C₆)haloalkyl, (C₃-C₈)cycloalkyl, (C₁-C₆)alkylaryl, heterocyclyl, aryl or heteroaryl, wherein R^{Z6} is optionally substituted with at least one R^{Z6′},
      wherein each Rz⁶′ is independently halogen, —OR, —C(O)OR, —C(O)R, (C₁-C₆)alkyl or (C₁-C₆)haloalkyl,
         wherein R^{Z6′} is optionally substituted with one or more R′.

In another embodiment, the disclosure provides compounds of formula (I) wherein
R^{N′} is hydrogen, (C₁-C₆)alkyl, (C₁-C₆)haloalkyl, (C₁-C₆)alkanoyl, (C₃-C₈)cycloalkyl, aryl, heteroaryl, (C₃-C₈)cycloalkanoyl, heterocycloyl, aroyl, heteroaroyl, (C₁-C₆)alkoxycarbonyl or aryl(C₁-C₆)alkoxycarbonyl, wherein
   R^{N′} is optionally substituted with one or more groups which are independently halogen, —OR^{N″}, —NR^{N″}₂, —NO₂, —CN, (C₁-C₆)alkyl, aryl, heterocyclyl, heteroaryl, (C₃-C₈)cycloalkyl or (C₁-C₆)haloalkyl,
      wherein each R^{N″} is independently hydrogen, (C₁-C₆)alkyl, (C₁-C₆)haloalkyl, (C₁-C₆)alkoxy, (C₃-C₈)cycloalkyl, heterocycloalkyl, aryl or heteroaryl, wherein the alkyl and alkoxy are optionally substituted with one or more R′.

In another embodiment, the disclosure provides compounds of formula (I) wherein B is not aromatic and G¹, G², and G³ are each independently O, N, CR³, C(R³)₂ or N(R^{N′}).

In another embodiment, the disclosure provides compounds of formula (I) wherein B is pyrrolidinyl, pyrazolidinyl, imidazolidinyl, isoxazolidinyl, oxazolidinyl, thiazolidinyl or tetrazolidinyl, wherein each carbon is substituted by two R³ and each nitrogen is substituted by R^{N′}.

In another embodiment, the disclosure provides compounds of formula (I) wherein
each R³ is independently R^{Z3}, wherein
   R^{Z3} is hydrogen, (C₁-C₆)alkyl, (C₁-C₆)haloalkyl, (C₃-C₈)cycloalkyl, (C₁-C₆)alkylaryl, heterocyclyl, aryl or heteroaryl, wherein R^{Z3} is optionally substituted with at least one R^{Z3′}, wherein
      each R^{Z3′} is independently halogen, —CN, —OR, —C(O)OR, —C(O)R, —C(O)NR₂, (C₁-C₆)alkyl, (C₁-C₆)haloalkyl, (C₃-C₈)cycloalkyl or heterocycloalkyl.

In another embodiment, the disclosure provides compounds of formula (I) wherein
R² and R⁶ are each hydrogen, halogen, —NO₂, —CN or —R^{Z6} wherein
   R^{Z6} is (C₁-C₆)alkyl, (C₁-C₆)haloalkyl, (C₃-C₈)cycloalkyl, (C₁-C₆)alkylaryl, heterocyclyl, aryl or heteroaryl, wherein R^{Z6} is optionally substituted with at least one R^{Z6′},
      wherein each Rz^{6′} is independently halogen, —OR, —C(O)OR, —C(O)R, (C₁-C₆)alkyl or (C₁-C₆)haloalkyl,
         wherein R^{Z6′} is optionally substituted with one or more R′.

In another embodiment, the disclosure provides compounds of formula (I) wherein
each R^{N′} is independently hydrogen, (C₁-C₆)alkyl, (C₁-C₆)haloalkyl, (C₁-C₆)alkanoyl, (C₃-C₈)cycloalkyl, aryl, heteroaryl, (C₃-C₈)cycloalkanoyl, heterocycloyl, aroyl, heteroaroyl, (C₁-C₆)alkoxycarbonyl or aryl(C₁-C₆)alkoxycarbonyl, wherein
   R^{N′} is optionally substituted with one or more groups which are independently halogen, —OR^{N″}, —NR^{N″}₂, —NO₂, —CN, (C₁-C₆)alkyl, aryl, heterocyclyl, heteroaryl, (C₃-C₈)cycloalkyl or (C₁-C₆)haloalkyl,
      wherein each R^{N″} is independently hydrogen, (C₁-C₆)alkyl, (C₁-C₆)haloalkyl, (C₁-C₆)alkoxy, (C₃-C₈)cycloalkyl, heterocycloalkyl, aryl or heteroaryl, wherein the alkyl and alkoxy are optionally substituted with one or more R′.

In another embodiment, the disclosure provides compounds of formula (I) wherein the compound of formula (I) is

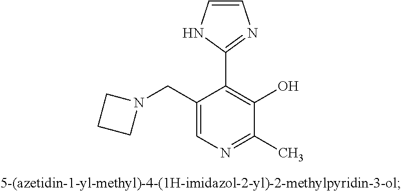 [SMILES: Cc1ncc(CN2CCC2)c(-c2ncc[nH]2)c1O]

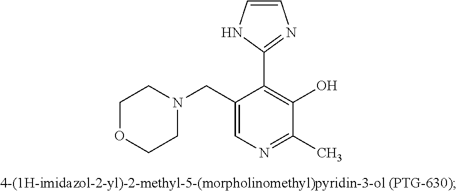 [SMILES: Cc1ncc(CN2CCOCC2)c(-c2ncc[nH]2)c1O]

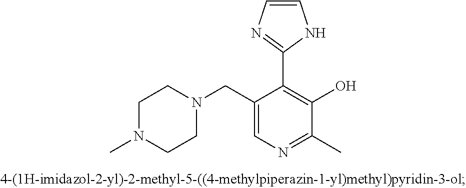

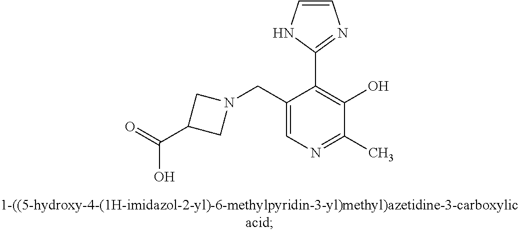 [SMILES: Cc1ncc(CN2CC(C(=O)O)C2)c(-c2ncc[nH]2)c1O]

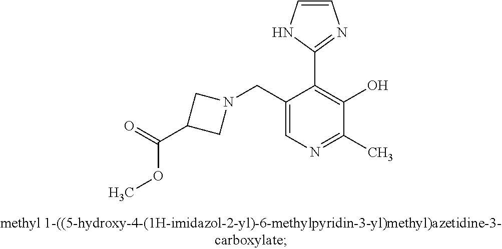 [SMILES: COC(=O)C1CN(Cc2cnc(C)c(O)c2-c2ncc[nH]2)C1]

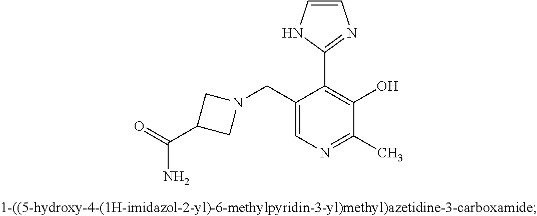 [SMILES: Cc1ncc(CN2CC(C(N)=O)C2)c(-c2ncc[nH]2)c1O]

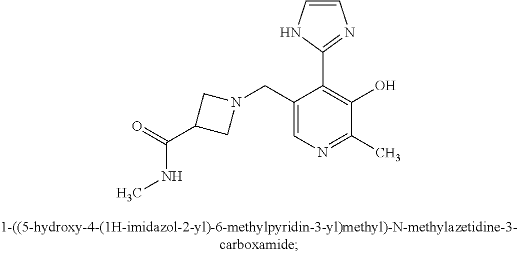 [SMILES: CNC(=O)C1CN(Cc2cnc(C)c(O)c2-c2ncc[nH]2)C1]

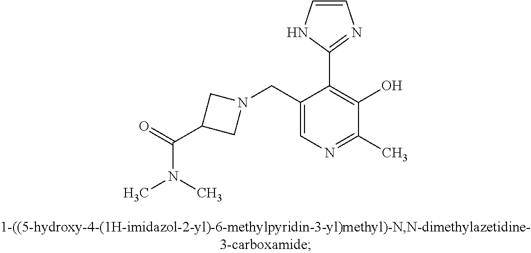 [SMILES: Cc1ncc(CN2CC(C(=O)N(C)C)C2)c(-c2ncc[nH]2)c1O]

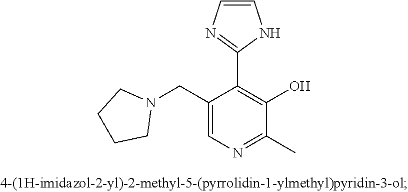 [SMILES: Cc1ncc(CN2CCCC2)c(-c2ncc[nH]2)c1O]

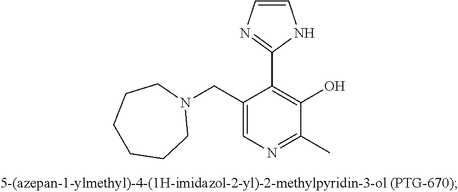 [SMILES: Cc1ncc(CN2CCCCCC2)c(-c2ncc[nH]2)c1O]

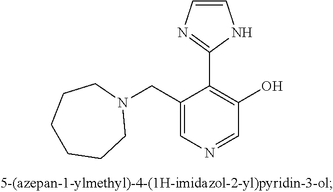 [SMILES: Oc1cncc(CN2CCCCCC2)c1-c1ncc[nH]1]

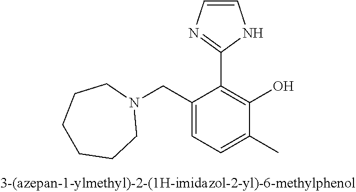 [SMILES: Cc1ccc(CN2CCCCCC2)c(-c2ncc[nH]2)c1O]

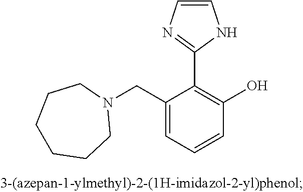 [SMILES: Oc1cccc(CN2CCCCCC2)c1-c1ncc[nH]1]

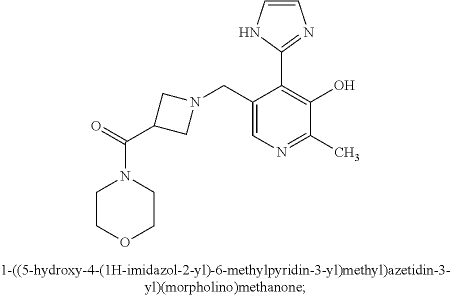 [SMILES: Cc1ncc(CN2CC(C(=O)N3CCOCC3)C2)c(-c2ncc[nH]2)c1O]

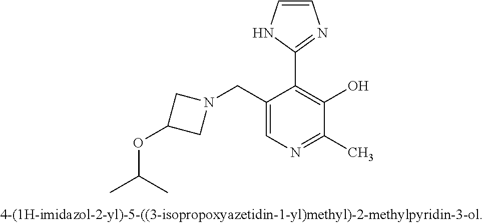 [SMILES: Cc1ncc(CN2CC(OC(C)C)C2)c(-c2ncc[nH]2)c1O]

or a pharmaceutically acceptable salt thereof.

In another aspect, the disclosure provides compounds that are:

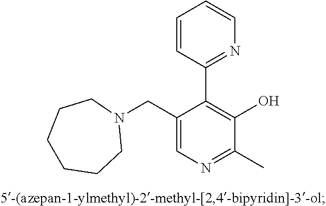 [SMILES: Cc1ncc(CN2CCCCCC2)c(-c2ccccn2)c1O]

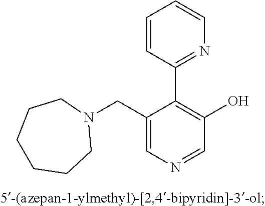 [SMILES: Oc1cncc(CN2CCCCCC2)c1-c1ccccn1]

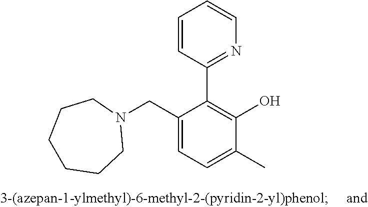 [SMILES: Cc1ccc(CN2CCCCCC2)c(-c2ccccn2)c1O]

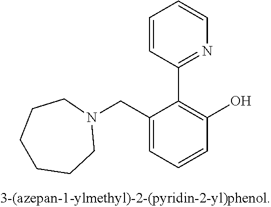 [SMILES: Oc1cccc(CN2CCCCCC2)c1-c1ccccn1]

or a pharmaceutically acceptable salt thereof.

In a specific embodiment, the compound comprises 4-(1H-imidazol-2-yl)-2-methyl-5-(morpholinomethyl)pyridin-3-ol (also referred to as PTG-630), 5-(azepan-1-ylmethyl)-4-(1H-imidazol-2-yl)-2-methylpyridin-3-ol (also referred to as PTG-670) or a pharmaceutically acceptable salt thereof.

### Definitions

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, the term “inhibiting development of” means to prevent or to minimize development of the disorder or complication in individuals at risk of developing the disorder or complication.

The term “absent” as used herein means the group is replaced by a single bond. If replacing the group with a bond results in two connected moieties both defined as bonds, then -bond-bond- groups are understood to reduce to a single bond.

The term “alkenyl” as used herein, means a straight or branched chain hydrocarbon containing from 2 to 10 carbons and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkanoyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means phenyl or a bicyclic aryl or a tricyclic aryl. The bicyclic aryl is naphthyl, or a phenyl fused to a cycloalkyl, or a phenyl fused to a cycloalkenyl. The bicyclic aryl is attached to the parent molecular moiety through any carbon atom contained within the bicyclic aryl. Representative examples of the bicyclic aryl include, but are not limited to, dihydroindenyl, indenyl, naphthyl, dihydronaphthalenyl, and tetrahydronaphthalenyl. The tricyclic aryl is anthracene or phenanthrene, or a bicyclic aryl fused to a cycloalkyl, or a bicyclic aryl fused to a cycloalkenyl, or a bicyclic aryl fused to a phenyl. The tricyclic aryl is attached to the parent molecular moiety through any carbon atom contained within the tricyclic aryl. Representative examples of tricyclic aryl ring include, but are not limited to, azulenyl, dihydroanthracenyl, fluorenyl, and tetrahydrophenanthrenyl.

The term “arylalkoxycarbonyl” as used herein, means an arylalkoxy group, as defined herein, appended to the parent molecular moiety through a carbonyl group, as defined herein. Representative examples of arylalkoxycarbonyl include, but are not limited to, benzyloxycarbonyl and naphth-2-ylmethoxycarbonyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “aroyl” as used herein, means an aryl group, as defined herein, appended to the parent molecular moiety through a carbonyl group, as defined herein. Representative examples of arylcarbonyl include, but are not limited to, benzoyl and naphthoyl.

The term “cycloalkyl” as used herein, means a saturated cyclic hydrocarbon group containing from 3 to 8 carbons, examples of cycloalkyl include cyclopropyl, cyclobutyl, cyclopentyl, cyclohexyl, cycloheptyl, and cyclooctyl.

The term “cycloalkanoyl” as used herein, means cycloalkyl group, as defined herein, appended to the parent molecular moiety through a carbonyl group, as defined herein. Representative examples of cycloalkylcarbonyl include, but are not limited to, cyclopropylcarbonyl, 2-cyclobutylcarbonyl, and cyclohexylcarbonyl.

The term “halo” or “halogen” as used herein, means —Cl, —Br, —I or —F.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, means a monocyclic heteroaryl or a bicyclic heteroaryl. The monocyclic heteroaryl is a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any nitrogen atom contained within the bicyclic heteroaryl. Representative examples of bicyclic heteroaryl include, but are not limited to, benzimidazolyl, benzofuranyl, benzothienyl, benzoxadiazolyl, cinnolinyl, dihydroquinolinyl, dihydroisoquinolinyl, furopyridinyl, indazolyl, indolyl, isoquinolinyl, naphthyridinyl, quinolinyl, tetrahydroquinolinyl, and thienopyridinyl.

The term “heteroaryloyl” as used herein, means a heteroaryl group, as defined herein, appended to the parent molecular moiety through a carbonyl group, as defined herein. Representative examples of heteroarylcarbonyl include, but are not limited to, fur-3-ylcarbonyl, 1H-imidazol-2-ylcarbonyl, 1H-imidazol-4-ylcarbonyl, pyridin-3-ylcarbonyl, 6-chloropyridin-3-ylcarbonyl, pyridin-4-ylcarbonyl, (6-(trifluoromethyl)pyridin-3-yl)carbonyl, (6-(cyano)pyridin-3-yl)carbonyl, (2-(cyano)pyridin-4-yl)carbonyl, (5-(cyano)pyridin-2-yl)carbonyl, (2-(chloro)pyridin-4-yl)carbonyl, pyrimidin-5-ylcarbonyl, pyrimidin-2-ylcarbonyl, thien-2-ylcarbonyl, and thien-3-ylcarbonyl.

The term “heterocycle” as used herein, means a monocyclic, and 3, 4, 5, 6 or 7 membered ring containing at least one heteroatom independently selected from the group consisting of O, N, and S. The 3 or 4 membered ring contains 1 heteroatom selected from the group consisting of O, N and S. The 5 membered ring contains zero or one double bond and one, two or three heteroatoms selected from the group consisting of O, N and S. The 6 or 7 membered ring contains zero, one or two double bonds and one, two or three heteroatoms selected from the group consisting of O, N and S. The heterocycle is connected to the parent molecular moiety through any carbon atom or any nitrogen atom contained within the monocyclic heterocycle. Representative examples of heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

The term “heterocycloyl” as used herein, means a heterocycle, as defined herein, appended to the parent molecular moiety through a carbonyl group, as defined herein.

The term “oxide” as used herein, means an —O moiety; for example, attachment of an oxide group to a nitrogen forms an N-oxide compound, as is familiar to those skilled in the art. In such compounds, the oxygen has a formal negative charge and the nitrogen has a formal positive charge, therefore, the entire compound has a zero net charge.

The term “oxo” as used herein, means an ═O moiety.

### Pharmaceutical Compositions and Administration

The instant compounds can be administered individually or in combination, usually in the form of a pharmaceutical composition. Such compositions are prepared in a manner well known in the pharmaceutical art and comprise at least one active compound.

As used herein, “pharmaceutically acceptable salts” are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds. The term “salts” refers to the relatively non-toxic, inorganic and organic acid addition salts. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. These may include chlorine, sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977; 66:1-19 which is incorporated herein by reference.) In one embodiment, the compound is in the form of a chloride salt, such as a mono hydrochloride (Cl⁻) salt. In another embodiment, the compound is in the form of a dihydrochloride or a trihydrochloride salt.

The compounds of the disclosure can be administered as the sole active pharmaceutical agent, or they can be used in combination with one or more other compounds useful for carrying out the methods of the disclosure, including but not limited to pyridoxamine, aminoguanidine, and compounds disclosed in WO 2004/019889 (including but not limited to BST 4996, BST 4997, and BST-146). When administered as a combination, the therapeutic agents can be formulated as separate compositions that are given at the same time or different times, or the therapeutic agents can be given as a single composition.

The compounds may be made up in a solid form (including granules, powders or suppositories) or in a liquid form (e.g., solutions, suspensions, or emulsions). The compounds of the disclosure may be applied in a variety of solutions and may be subjected to conventional pharmaceutical operations such as sterilization and/or may contain conventional adjuvants, such as preservatives, stabilizers, wetting agents, emulsifiers, buffers etc.

The compounds of the disclosure may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of the disclosure and a pharmaceutically acceptable carrier. One or more compounds of the disclosure may be present in association with one or more non-toxic pharmaceutically acceptable carriers and/or diluents and/or adjuvants, and if desired other active ingredients. The pharmaceutical compositions containing compounds of the disclosure may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the disclosure may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds and pharmaceutical compositions of the present disclosure may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds and pharmaceutical compositions of the present disclosure may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.01 mg to about 50 mg per kilogram of body weight per day, more preferably between 0.1 mg to about 50 mg per kilogram of body weight per day, and even more preferably between about 0.1 mg to about 20 mg per kilogram of body weight per day are useful in the treatment of the above-indicated conditions. The amount of active ingredient that may be combined with the carrier materials to produce a single dosage form will vary depending upon the host treated and the particular mode of administration. Dosage unit forms will generally contain between from about 1 mg to about 500 mg of an active ingredient. The active compounds are effective over a wide dosage range. However, it will be understood that the amount of the compound actually administered will be determined by a physician, in the light of the above relevant circumstances. Therefore, the above dosage ranges are not intended to limit the scope of the invention in any way.

Pharmaceutical compositions containing the compounds described herein are administered to an individual in need thereof. In a preferred embodiment, the subject is a mammal; in a more preferred embodiment, the subject is a human. In therapeutic applications, compositions are administered in an amount sufficient to carry out the methods of the disclosure. Amounts effective for these uses depend on factors including, but not limited to, the nature of the compound (specific activity, etc.), the route of administration, the stage and severity of the disorder, the weight and general state of health of the subject, and the judgment of the prescribing physician. The active compounds are effective over a wide dosage range. However, it will be understood that the amount of the compound actually administered will be determined by a physician, in the light of the above relevant circumstances. Therefore, the above dosage ranges are not intended to limit the scope of the disclosure in any way.

For administration to non-human mammals, the composition may also be added to the animal feed or drinking water. It may be convenient to formulate these animal feed and drinking water compositions so that the animal ingests an appropriate quantity of the composition during a meal or throughout the course of the day. It may also be convenient to present the composition as a premix for addition to the feed or drinking water.

Detailed disclosure relating to the compounds for use in the methods of the disclosure, and methods for making the compounds, are disclosed in published PCT application WO2021022215, incorporated by reference herein in its entirety.

### EXAMPLES

### Example 1

### Objectives: To Assess the Efficacy of PTG-630 Administration in a Model of Alzheimer's Disease, the Human Tau Mice.

The human tau mouse is a model of sporadic dementia that expresses human non-mutant tau (6 isoforms) in the absence of endogenous mouse tau. The model develops age-associated pathology, with hyperphosphorylated tau detected in cell bodies and dendrites by 3 months of age (Andorfer et al., 2003; J Neurochem 86, 582-590, doi:10.1046/j.1471-4159.2003.01879.x). These mice display abnormal spatial learning by 6 months of age and impaired learning in the Morris water maze with memory loss by 12 months of age, as well as memory deficits in the Barnes maze by 6 months of age (Marquez et al, 2021).

Female and male human tau mice (Jax #005491) were treated daily for 25 weeks with PTG-630 trihydrochloride at 1 and 5 mg/kg in drinking water starting at 10 weeks of age (before cognitive deficits appear).

### Groups:

1—Control (C₅₇B16), N=7 male, N=7 female, Final N=6 male, N=6 female
2—htau, N=7 male, N=7 female, Final N=7 male, N=6 female
3—htau+PTG-630 1 mg/kg drinking water, N=7 male, N=7 female, Final N=7 male, N=7 female
4—htau+PTG-630 5 mg/kg drinking water, N=7 male, N=7 female, Final N=6 male, N=7 female

No mice died due to treatment. One mouse in group 4 died at week 8 from tooth occlusion. No toxicity was apparent.

### Assays:

Learning and memory were assessed using the Barnes maze test and the object recognition test at 6-8, 12-13, 18-19 and 24-25 weeks. Corneal nerves were imaged by corneal confocal microscopy (CCM) at week 6, 9, 12, 18 and 25. Data represent male and female results combined, mean±sem.

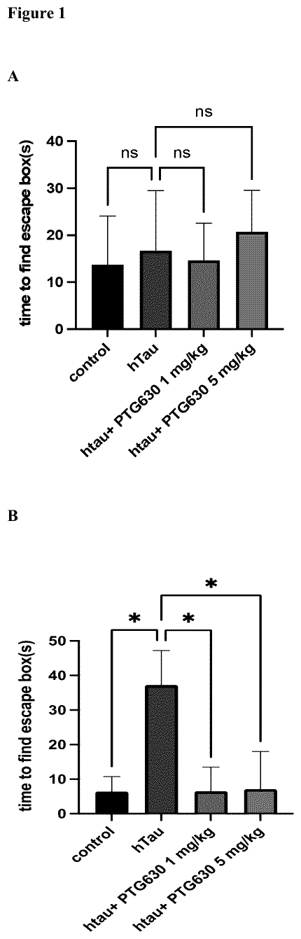

Using the Barnes maze test, long-term memory was assessed between week 13 and 19 and between week 19 and 23 of treatment (FIG. 1A, B). Human tau mice develop a significant memory impairment by week 23 of treatment, corresponding to 8 months of age, that was significantly prevented by treatment with PTG-630 (FIG. 1B).

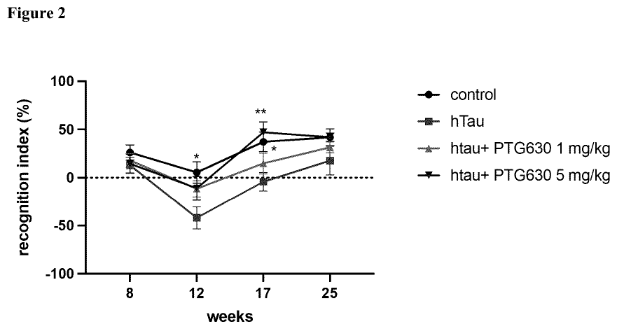

Similarly and as previously published (Marquez et al, 2021), human tau mice presented short term episodic memory deficits in the Object recognition test that was partially ameliorated by treatment with PTG-630 at 1 mg/kg and significantly alleviated after treatment with PTG-630 at 5 mg/kg for 17 weeks (FIG. 2).

Western blot Analysis

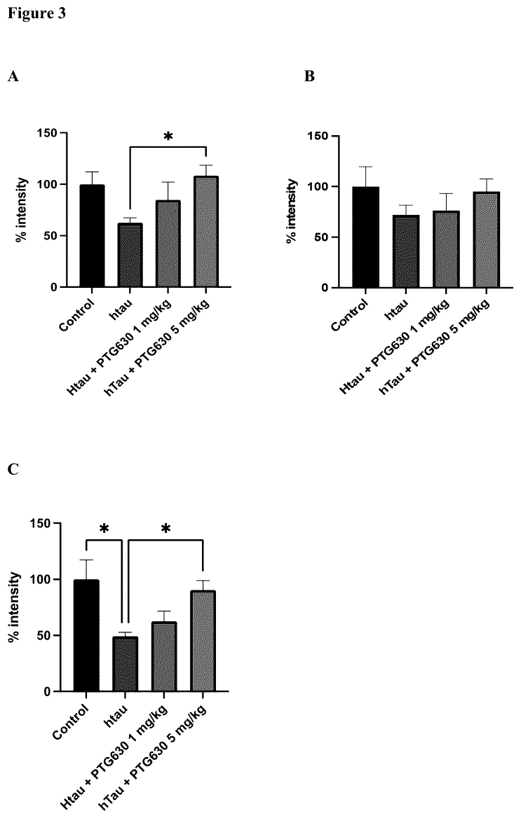

Mouse Hippocampus were processed for Western blotting analysis of (A) Drebrin (actin binding protein in dendritic spines, role in neuronal growth, a decrease=contributing factor in the pathogenesis of memory disturbance), (B) PSD95 (a post-synaptic protein, role in the regulation of excitatory synapse maturity), and (C) NeuN (neuronal nuclear antigen, biomarker for neurons, decrease=neuronal loss) (FIG. 3). The reduced drebrin levels in the hippocampus of human tau mice was significantly (p<0.05) ameliorated by 12 weeks of treatment with PTG-630 in drinking water. Similarly, levels of PSD95 protein tend to decrease in human tau mice and to be dose-dependly ameliorate by treatment with PTG-630. A significant neuronal loss (p<0.05) in human tau mouse hippocampus was partially alleviated by treatment with PTG-630 at 5 mg/kg.

### Tactile and Thermal Responses

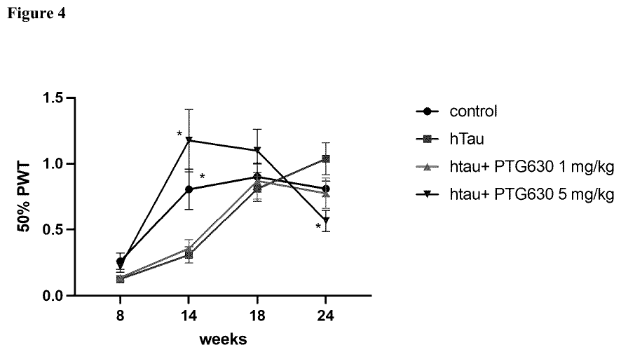

Tactile and thermal responses, measures of associated damage to the peripheral nervous system (PNS), were assessed at 8, 14, 18 and 24 weeks. hTau mice developed significant tactile allodynia measured with manual von Frey filaments at the 14 weeks time point (FIG. 4). Treatment with PTG-630 significantly ameliorated tactile allodynia in human tau mice after 14 weeks of treatment with 5 mg/kg PTG-630 (FIG. 4).

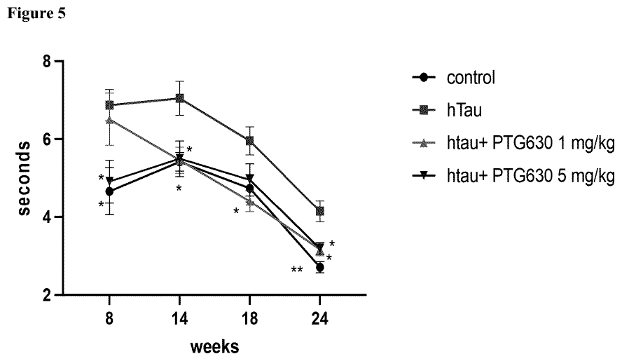

Human tau mice develop significant hypoalgesia (FIG. 5). PTG-630 significantly ameliorated thermal hypoalgesia (FIG. 5) at 5 mg/kg by 8 weeks of treatment and for the duration of the study. The effect of PTG-630 at 1 mg/kg was significant by 14 weeks of treatment and remained for the rest of the study (FIG. 5).

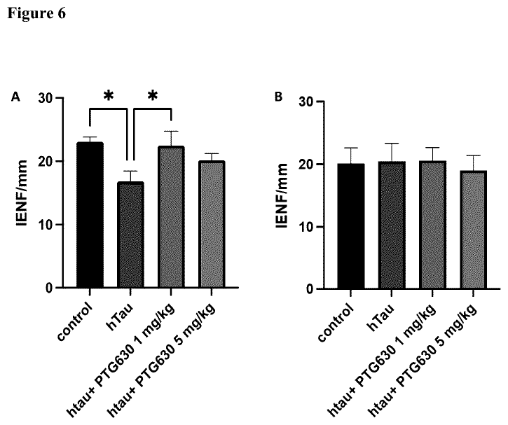

A standard measure of associated PNS damage to neuronal tissue and its amelioration is IENF, seen alongside changes in the CNS. Quantification of intraepidermal fibers (IENF) in the footskin of htau mice showed a significant (p<0.05) decreased of IENF in male htau skin that was significantly (p<0.05) ameliorated by 124 weeks of treatment with PTG-630 at 1 mg/kg (FIG. 6A). No difference was noted for female mice (FIG. 6B). The amelioration of these PNS effects confirms that PTG-630 readily enters and targets neuronal tissue, whether in the brain or elsewhere in the body.

### Other Assays

Motor nerve conduction velocity (NCV) was measured at 8, 16 and 25 weeks after treatment. Nerve conduction velocity was not significantly different from control levels in htau mice with or without treatment with PTG-630 (data not shown).

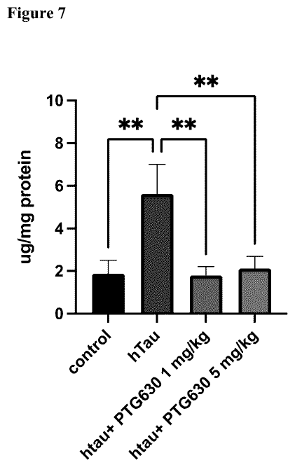

Lipid peroxidation was assessed using the 4-hydroxynonal (4-HNE) Oxiselect ELISA in hippocampus homogenates. 4-HNE levels were significantly (p<0.001) increased in htau hippocampus and treatment with 1 and 5 mg/kg PTG-630 significantly (p<0.01) reduced HINE levels to control levels (FIG. 7). It is believed that damage to neuronal tissue occurs to a significant extent via induction of oxidative stress that leads to lipoxidation, so that amelioration by PTG-630 reflects their efficacy via anti-oxidant mechanisms.

Terminal plasma and brain cortex were also analyzed for carboxymethyl Lysine (CML), a prominent advanced glycation end-product (AGE), by an LC-MS/MS quantitative chemical analysis method. PTG-630 and PTG-670 were developed as potent AGE inhibitors and tested for amelioration of PNS and CNS damage due to the extensive work that implicated AGE formation as a causative factor in the development of neurodegenerative pathologies. CML levels were significantly (p<−0.05) increased in cortex from female htau mice and significantly (p<0.05) reduced by treatment with PTG-630 at 5 mg/kg. No significant difference was observed in plasma (male and female) and in male cortex (data not shown).

### Blood Brain Barrier (BBB) Penetration

This was a study in male Sprague-Dawley rats. The primary objective was to assess the IV and PO pharmacokinetics of PTG-630. The secondary objectives were to assess tissue distribution after oral administration of PTG-630 (free base form) and the renal excretion, hepatic extraction efficiency and bioavailability of PTG-630.

Twelve non-cannulated animals were arbitrarily assigned to groups upon arrival and housed together throughout the experiment. The animals were acclimated to their designated housing for at least 24 hours before the first day of dosing. PTG-630 was formulated in 0.5% methyl cellulose and 0.1% Tween 20 in water for injection and administered to animals by mouth (PO) with blood and tissue samples collected at predetermined time points. Right thigh muscle, liver, brain, right sciatic nerve, and right kidney were collected at the terminal time points. These samples were collected and placed into individually labeled tubes and frozen at −20° C. Tissues samples were analyzed for concentration of PTG-630. The data is shown in Table 1.

TABLE 1
Pharmacokinetics of PTG-630

| | | | Time (h) | | | | | |
|---|---|---|---|---|---|---|---|---|
| | | | 0.50 | 1.0 | 2.0 | 4.0 | 6.0 | 24 |
| Analyte | matrix | | Concentration (ng/mL) | | | | | |
| PTG-630 | Plasma | Mean | 930 | 1160 | 274 | 794 | 510 | 18.4 |
| PTG-630 | Brain | Mean | 1430 | 1990 | 450 | 1000 | 244 | BLQ |
| PTG-630 | Sciatic | Mean | 1660 | 1830 | 510 | 989 | 690 | BLQ |
| | Nerve | | | | | | | |
| PTG-630 | Muscle | Mean | 775 | 1030 | 290 | 401 | 245 | BLQ |
| PTG-630 | Kidney | Mean | 2230 | 3880 | 1510 | 1520 | 466 | BLQ |
| PTG-630 | Liver | Mean | 5720 | 6350 | 5080 | 2420 | 647 | BLQ |

The pharmacokinetics data was analyzed to extract standard parameters, shown in Table 2.

TABLE 2
Pharmacokinetics of PTG-630: Standard Parameters

| | | Tmax | Cmax | Cmax (SE) | AUClast | AUClast (SE) | AUCinf | % Extrap |
|---|---|---|---|---|---|---|---|---|
| Analyte | matrix | (h) | (ng/mL) | (ng/mL) | (h*ng/mL) | (h*ng/mL) | (h*ng/mL) | (%) |
| PTG-630 | Brain | 1.0 | 1990 | 300 | 5130 | 928 | 6720 | 23.7 |
| | Kidney | 1.0 | 3880 | 655 | 9770 | 673 | 11400 | 14.0 |
| | Liver | 1.0 | 6350 | 1010 | 20700 | 2010 | 22000 | 5.72 |
| | Muscle | 1.0 | 1030 | 65.5 | 2650 | 330 | 3820 | 30.7 |
| | Plasma | 1.0 | 1160 | 168 | 8600 | 804 | 8700 | 1.13 |
| | Sciatic Nerve | 1.0 | 1830 | 200 | 6550 | 394 | NC | NC |

PTG-630 was quantifiable in all tested tissues (liver, kidney, brain, sciatic nerve, muscle). The exposure pattern of PTG-630 in tissues was comparable to those in plasma with Tmax 1 hour. The level of PTG-630 exposure in selected tissues exceeds the exposure in plasma up to 3-5 folds. PTG-630 was not detectable in any tissue, except plasma 24 hours post dose. Significant exposure of PTG-630 in brain and sciatic nerve was determined, with Cmax 1990 ng/mL for brain and 1830 ng/mL for sciatic nerve, indicating blood brain permeability for compound PTG-630. Half-life of PTG-630 in brain was 4.53 hours, which is 1.3 times higher than in plasma.

### Example 2

### Objectives: To Assess the Efficacy of PTG-670 Administration in a Murine Model of Alzheimer's Disease (AD), the 3×Tg or LaFerla Mice.

The 3×Tg mouse carries 3 mutations associated with familial AD: amyloid precursor protein (APP) Swedish mutation, microtubule-associated protein tau (MAPT) P301L and presenilin 1 (PSEN1) M146V (Oddo et al, 2003). Mice develop both plaque and tangle pathology, with a progressive deposition of Aβ as early as 3-4 months of age. Cognitive impairments manifest as early as 4-5 months of age, with initial onset of memory retention deficits followed by onset of learning deficits in the Barnes maze test (Stover et al., 2015). There is increased lipoperoxidation and reduced superoxide dismutase (SOD), an anti-oxidant enzyme, in 3×Tg mice (Garcia-Mesa et al., 2016) and increased so-called receptor for advanced glycation end-products (AGE) (RAGE) expression in older animals (Choi et al., 2014).

Female and male 3×Tg mice (MMRRC #034830) were treated daily for 20 weeks with PTG-670 trihydrochloride at 1 and 5 mg/kg in drinking water starting at 19 weeks of age (˜5 months old) (just before cognitive deficits onset).

### Groups:

1—Control (B6129SF2/J Jax 101045)), N=7 male, N=7 female,
2—3×Tg, N=7 male, N=7 female,
3—3×Tg+PTG-670 1 mg/kg drinking water, N=7 male, N=7 female,
4—3×Tg+PTG-670 5 mg/kg drinking water, N=7 male, N=7 female.

No mice died during the course of the study. No apparent toxicity was observed.

### Assays:

Tactile and thermal responses were assessed at 5 and 11 weeks. Motor nerve conduction velocity (NCV) was measured at 8, 12 and 20 weeks after treatment. Learning and memory were assessed using the Barnes maze test and the object recognition test at 6, 12, and 18 weeks of treatment. Corneal nerves were imaged by corneal confocal microscopy (CCM) at week 5, 11 and 17. Data represent male and female results combined, unless specified, mean±sem.

### Peripheral Nervous System (PNS):

3×Tg mice did not develop tactile allodynia measured with manual von Frey filaments over the 20 weeks of the study (data not shown). Treatment with PTG-670 did not affect mice responses to von Frey filaments. 3×Tg mice did not develop significant hypoalgesia up to 9 months of age. PTG-670 did not affect the 3×Tg mice responses to heat stimuli (data not shown).

Male 3×Tg mice develop significant (p<0.05) nerve conduction velocity slowing by 9 months of age (20 weeks time point) (data not shown). The slowing of NCV was prevented by treatment with PTG-670 at both 1 and 5 mg/kg in drinking water. Female 3×Tg mice did not develop NCV slowing (data not shown).

### Central Nervous System (CNS):

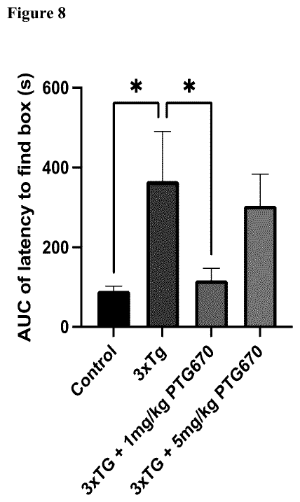

Learning abilities assessed using the Barnes maze were impaired in 3×Tg mice in both male and female around 6-7 months, conform with the literature (Stover et al., 2015). Treatment with PTG-670 did not improve learning deficits in the male 3×Tg mice (data not shown). Female 3×Tg mice treated with PTG-670 at 1 mg/kg for 18 weeks displayed significantly improved learning abilities to level similar to control (FIG. 8).

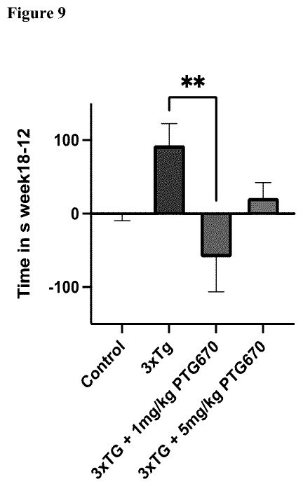

Using the Barnes maze test, long-term memory was assessed between week 12 and 18 of treatment. Female 3×Tg mice develop memory impairment by week 18 of treatment, corresponding to 9 months of age, that was significantly (p<0.05) prevented by treatment with PTG-670 at 1 mg/kg (FIG. 9). No difference in memory abilities or effect of PTG-670 were observed in the male 3×TG mice.

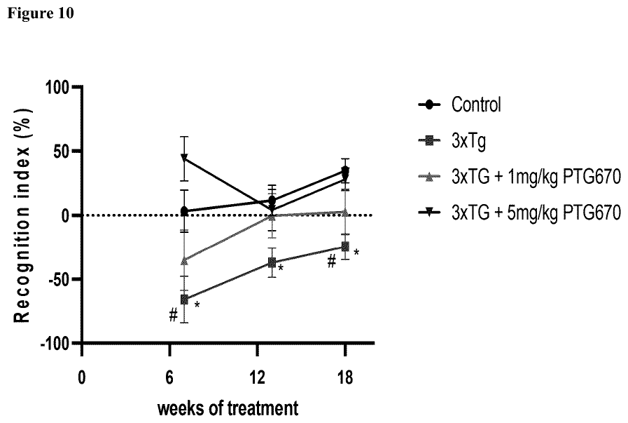

Similarly, 3×Tg mice presented significant short term episodic memory deficits in the Object Recognition test that was significantly (p<0.05) ameliorated by treatment with PTG-670 at 5 mg/kg after 6 and 18 weeks of treatment (FIG. 10).

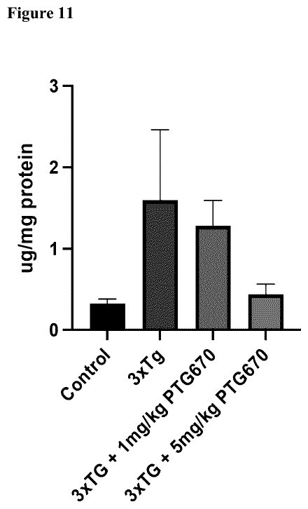

Lipid peroxidation was assessed using the 4-Hydroxynonal (4-HNE) Oxiselect Elisa in hippocampus homogenates. 4-HNE levels tend to increase in 3×Tg mouse hippocampus and treatment with 5 mg/kg PTG-670 tend to reduce HNE levels (FIG. 11).

### Western Blot Analysis

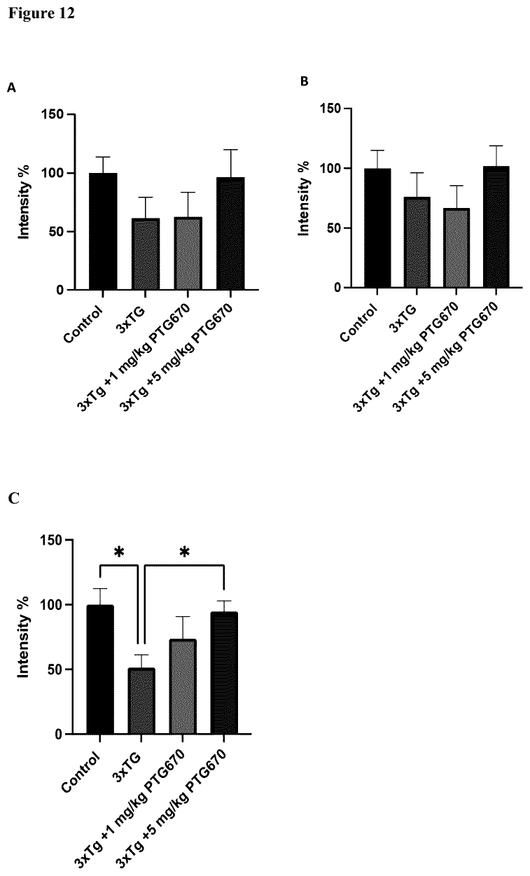

Mouse Hippocampus were processed for Western blotting analysis of Drebrin (actin binding protein in dendritic spines, role in neuronal growth, a decrease is a contributing factor in the pathogenesis of memory disturbance), PSD95 (a post-synaptic protein, role in the regulation of excitatory synapse maturity), and NeuN (neuronal nuclear antigen, biomarker for neurons, decrease=neuronal loss) (FIG. 12). The reduced drebrin levels in the hippocampus of 3×Tg mice was ameliorated by 20 weeks of treatment with PTG-670 at 5 mg/kg in drinking water only for female mice. Similarly, levels of PSD95 protein tend to decrease in 3×Tg mice and to be ameliorated by 5 mg/kg PTG670. A significant neuronal loss (p<0.05) in 3Tg mouse hippocampus was significantly (p<0.05) elevated by treatment with PTG-670 at 5 mg/kg only for female mice. No differences were observed for hippocampus from male 3×Tg mice.

1. A method for treating or inhibiting development of a disorder selected from the group consisting of a frontotemporal dementia (FTD), Pick's disease, progressive supranuclear palsy, Huntington's disease, Parkinson's disease, corticobasal degeneration, amyotrophic lateral sclerosis (ALS), Lewy body disease, and hippocampal sclerosis (HS), comprising administering to a subject an amount effective to treat or inhibit development of the disorder of a compound of formula I: 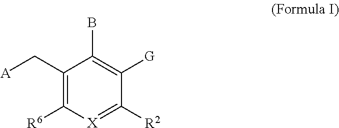
or a pharmaceutically acceptable salt thereof, wherein
X is N, N—O or CR¹;
G is —OH, —SH, —NH₂, or —N(R^{G})₂, wherein R^{G} is hydrogen, (C₁-C₆)alkyl or —C(O)(C₁-C₆)alkyl;
A is 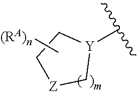
wherein
Y is N;
Z is CH₂, C(H)R^{A}, C(R^{A})₂, O, or NR^{A};
m is 0, 1, 2, or 3;
provided that
when m is 0, Z is CH₂, C(H)R^{A} or C(R^{A})₂, and
when m is 2, Z is O or NR^{A};
R^{A} is (C₁-C₆)alkyl, halogen, —OR^{A1}, —N(R^{A1})₂, —SR^{A1}, —S(O)R^{A1}, —S(O)₂R^{A1}, —COOR^{A1}, —CON(R^{A1})₂ or —(C₁-C₆)alkyl-OR^{A1},
wherein R^{A1} is hydrogen, (C₁-C₆)alkyl or —C(O)(C₁-C₆)alkyl, or two R^{A1} together with N-atom to which they are attached form a morpholinyl; and
n is 0, 1, 2, 3, 4, 5 or 6;
B is of the formula, 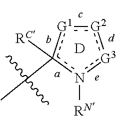
wherein
ring D is (i) monocyclic, and
(ii) unsaturated or aromatic;
R^{C′} is hydrogen;
G¹ is O, S, N or NR^{N′};
G² and G³ each are independently N, O, CR³, C(R³)₂ or NR^{N′}, wherein each R³ is independently —Z³-M-Z⁴—R^{Z}, wherein M is —C(O)—, —C(S)—, —S(O)—, —S(O)₂—, or absent,
Z³ and Z⁴ are independently —O—, —S—, —N(R^{N3})— or absent, wherein
R^{N3} is hydrogen, (C₁-C₆)alkyl, (C₂-C₆)alkenyl, (C₁-C₆)alkynyl, (C₁-C₆)haloalkyl, (C₁-C₆)alkanoyl, (C₃-C₈)cycloalkanoyl, heterocycloyl, aroyl, heteroaroyl, (C₁-C₆)alkoxycarbonyl or aryl(C₁-C₆)alkoxycarbonyl, wherein
R^{N3} is optionally substituted with one or more groups which are independently halogen, —OH, amino, (C₁-C₆)alkylamino, (C₁-C₆)dialkylamino, —NO₂, —CN, (C₁-C₆)alkyl, aryl, heterocyclyl, heteroaryl, (C₃-C₈)cycloalkyl, (C₁-C₆)haloalkyl, (C₁-C₆)alkoxy, (C₁-C₆)alkanoyl or aroyl;
R^{Z} is —H, —(C₁-C₆)alkyl, —(C₂-C₆)alkenyl, —(C₁-C₆)alkynyl, or —(C₁-C₆)haloalkyl, —(C₃-C₈)cycloalkyl, —(C₁-C₆)alkylaryl, -heterocycle, -aryl, or -heteroaryl, wherein
R^{Z} is optionally substituted with at least one R^{Z′}, wherein
each R^{Z′ is independently -halogen, —OR, —(C}₁-C₆)alkoxy, —C(O)OR, —C(O)R, —C(O)NR₂, —S(O)₂R, —OS(O)₂R, -cyano, -nitro, —(C₁-C₆)alkyl, —(C₁-C₆)haloalkyl, —(C₃-C₈)cycloalkyl, -heterocycloalkyl, or heteroaryl,
provided when M is —S(O)—, —S(O)₂— or absent, at least one of Z³ and Z⁴ is also absent;
or two R³ taken together are oxo;
R^{N′} is hydrogen or C₁-C₆)alkyl;
bonds a, b, c, d, and e are independently a single or double bond,
provided that
(i) no two consecutive atoms in ring D are both oxygen;
(ii) no two consecutive bonds are both double bonds;
(iii) if a or b is a double bond, then R^{C′} is absent; and
(iv) if a or e is a double bond, then R^{N′} is absent;
(v) if b or c is a double bond, then G¹ is not O or S;
(vi) if c or d is a double bond, then G² is not O;
(vii) if d or e is a double bond, then G³ is not O;
R¹, R², and R⁶ are independently hydrogen, halogen, —NO₂, —CN or R^{C},
provided that when X═CR¹,
(i) R², R⁶, and R^{N1} are not phenyl;
(ii) R^{C} is not aryl, heteroaryl, heterocyclyl or (C₂-C₆)alkenyl
(iii) and G¹=N together, then G₂ is not O; and
(iv) two R^{C} together may not form oxo;
and provided that when X═N, and
(i) G¹ is N, G³ is CR³ and G² is N, and bonds b and d are each a double bond, all simultaneously; or
(ii) G¹ is N, G³ is C(O), G² is NR^{N′}, and bond b is a double bond, all simultaneously;
either R² or R⁶ is not —NH-aryl or —NH-heteroaryl,
or a pharmaceutically acceptable salt thereof.

2. (canceled)

3. The method according to claim 1, wherein
wherein X is N and G is —OH;
B is aromatic; and
G¹ is O, S, N or NR^{N′}; and
G² and G³ are each independently O, N or CR³.

4. The method according to claim 3, wherein B is imidazolyl, oxazolyl, pyrazolyl, pyrrolyl or isoxazolyl wherein each carbon atom is substituted by R³.

5. The method according to claim 4, wherein B is imidazolyl wherein each carbon atom is substituted by R³.

6.-8. (canceled)

9. The method according to claim 5, wherein A is 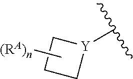
wherein n is 0, 1, 2 or 3.

10. The method according to claim 5, wherein A is

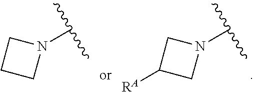

11. The method according to claim 5, wherein A is

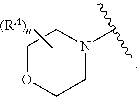

12.-44. (canceled)

45. The method according to claim 1, wherein the compound is selected from the group consisting of: 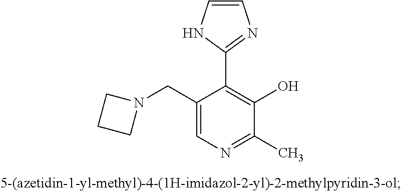 [SMILES: Cc1ncc(CN2CCC2)c(-c2ncc[nH]2)c1O] 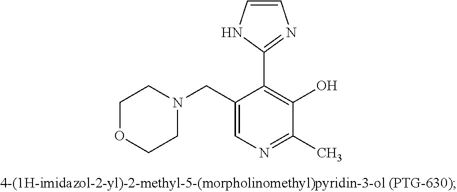 [SMILES: Cc1ncc(CN2CCOCC2)c(-c2ncc[nH]2)c1O] 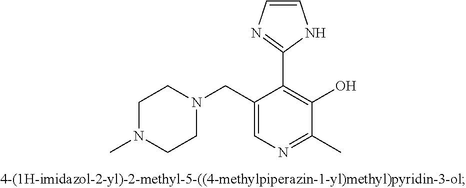 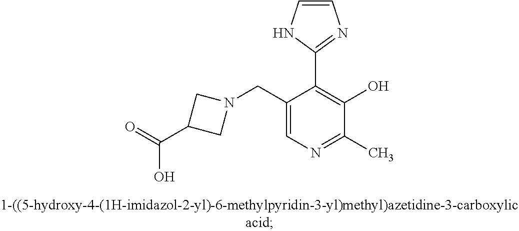 [SMILES: Cc1ncc(CN2CC(C(=O)O)C2)c(-c2ncc[nH]2)c1O] 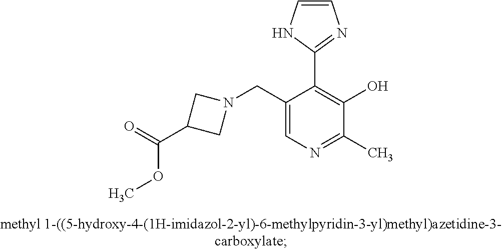 [SMILES: COC(=O)C1CN(Cc2cnc(C)c(O)c2-c2ncc[nH]2)C1] 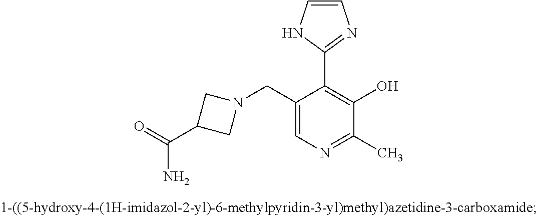 [SMILES: Cc1ncc(CN2CC(C(N)=O)C2)c(-c2ncc[nH]2)c1O] 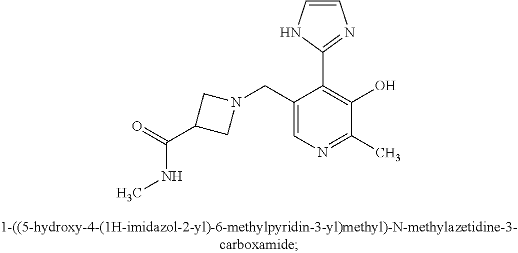 [SMILES: CNC(=O)C1CN(Cc2cnc(C)c(O)c2-c2ncc[nH]2)C1] 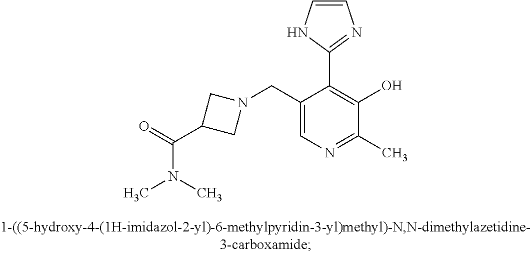 [SMILES: Cc1ncc(CN2CC(C(=O)N(C)C)C2)c(-c2ncc[nH]2)c1O] 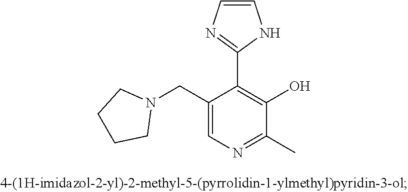 [SMILES: Cc1ncc(CN2CCCC2)c(-c2ncc[nH]2)c1O] 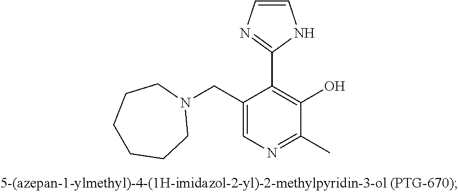 [SMILES: Cc1ncc(CN2CCCCCC2)c(-c2ncc[nH]2)c1O] 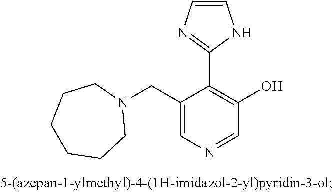 [SMILES: Oc1cncc(CN2CCCCCC2)c1-c1ncc[nH]1] 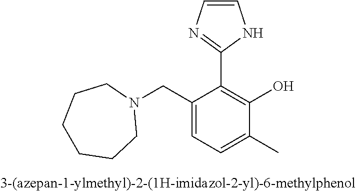 [SMILES: Cc1ccc(CN2CCCCCC2)c(-c2ncc[nH]2)c1O] 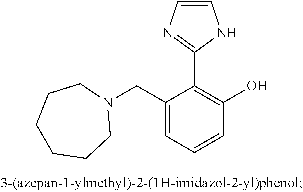 [SMILES: Oc1cccc(CN2CCCCCC2)c1-c1ncc[nH]1] 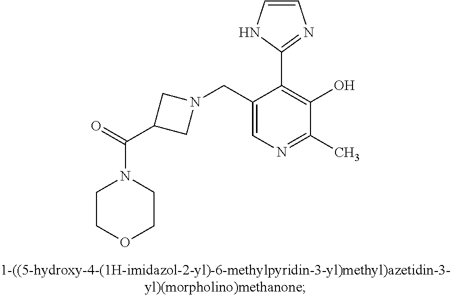 [SMILES: Cc1ncc(CN2CC(C(=O)N3CCOCC3)C2)c(-c2ncc[nH]2)c1O] 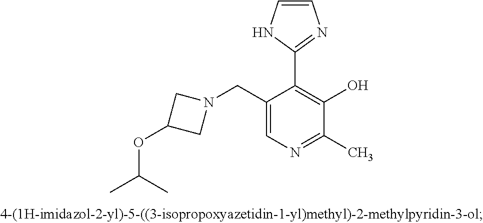 [SMILES: Cc1ncc(CN2CC(OC(C)C)C2)c(-c2ncc[nH]2)c1O]
or a pharmaceutically acceptable salt thereof.

46. The method according to claim 1, wherein the compound is selected from the group consisting of: 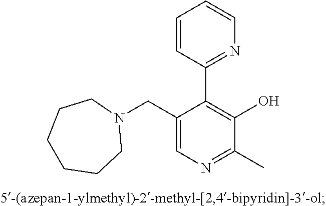 [SMILES: Cc1ncc(CN2CCCCCC2)c(-c2ccccn2)c1O] 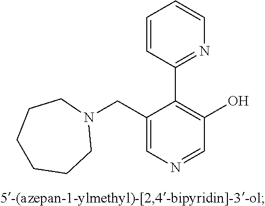 [SMILES: Oc1cncc(CN2CCCCCC2)c1-c1ccccn1] 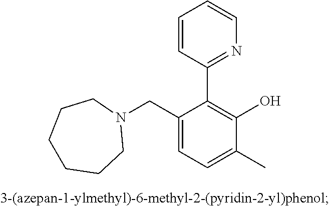 [SMILES: Cc1ccc(CN2CCCCCC2)c(-c2ccccn2)c1O] 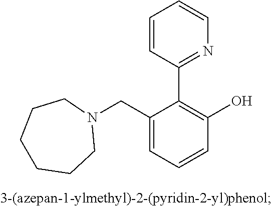 [SMILES: Oc1cccc(CN2CCCCCC2)c1-c1ccccn1]
or a pharmaceutically acceptable salt thereof.

47. The method according to claim 1, wherein the compound is in form of a pharmaceutically acceptable salt.

48. The method according to claim 1, wherein the compound is in form of a zinc (Zn²⁺) salt.

49. The method according to claim 48, wherein the zinc salt is ZnCl₂, ZnBr₂, ZnI₂, Zn(NO₃)₂, ZnSO₄, Zn₃(PO₄)₂, or Zn(OH)₂.

50. The method according to claim 48, wherein the compound is complexed with Zn²⁺ in a 1:1 stoichiometry.

51. The method according to claim 49, wherein the compound is complexed with Zn²⁺ in a 2:1 stoichiometry or a 3:1 stoichiometry.

52.-72. (canceled)

73. The method of claim 1, wherein the compound is administered orally.

74. The method of claim 1, wherein the compound is administered intravenously.

75. The method of claim 1, wherein the compound comprises 4-(1H-imidazol-2-yl)-2-methyl-5-(morpholinomethyl)pyridin-3-ol (also referred to as PTG-630), 5-(azepan-1-ylmethyl)-4-(1H-imidazol-2-yl)-2-methylpyridin-3-ol (also referred to as PTG-670) or a pharmaceutically acceptable salt thereof.

76. The method of claim 1, wherein the compound is in the form of a dihydrochloride or a trihydrochloride salt.

77. The method according to claim 1, wherein the compound is 5-(azepan-1-ylmethyl)-4-(1H-imidazol-2-yl)-2-methylpyridin-3-ol 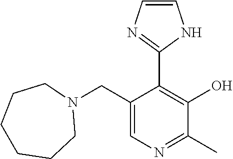 [SMILES: Cc1ncc(CN2CCCCCC2)c(-c2ncc[nH]2)c1O]
or a pharmaceutically acceptable salt thereof.

78. The method according to claim 1, wherein the compound is 4-(1H-imidazol-2-yl)-2-methyl-5-(morpholinomethyl)pyridin-3-ol 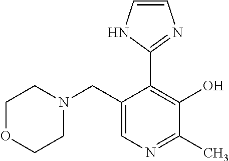 [SMILES: Cc1ncc(CN2CCOCC2)c(-c2ncc[nH]2)c1O]
or a pharmaceutically acceptable salt thereof.
